(19) United States
(12) Patent Application Publication
(10) Pub. No.: US 2007/0128644 A1
(43) Pub. Date: Jun. 7, 2007

(54) Fluid control method and fluid control apparatus

(75) Inventor: Katsumi Munenaka, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/604,811

(22) Filed: Nov. 28, 2006

(30) Foreign Application Priority Data
Dec. 1, 2005 (JP) 2005-348012

(51) Int. Cl. C12Q 1/68; C12M 1/34

(52) U.S. Cl. 435/6; 435/287.2; 435/288.5; 435/293.1

(57) ABSTRACT

The object of the present invention is to enable a probe to uniformly encounter biopolymers in a sample solution, regardless of a position in a reaction chamber. In the present invention, a fluid displacement between a reaction chamber and ports communicating therewith in a biochemical reaction part is controlled by switching control means formed of valves and syringe pumps. After a hybridization solution is poured into the reaction chamber, air is introduced through ports by valves, and is sucked from other ports by syringe pumps. The valves are suitably turned on and off to switch the flow of the hybridization solution in the reaction chamber to directions Y, A and B, thus executing agitation in various directions. Thus, each probe is made to more securely encounter the biopolymers present in the hybridization solution, thereby achieving a hybridized coupling more efficiently regardless of the position of the probe.

### BACKGROUND OF THE INVENTION

1. Field of the Invention

The present invention relates to a fluid control method and a fluid control apparatus for controlling a flow of a fluid such as a sample solution, a cleaning liquid or a gas (such as air) in a reaction chamber at least a part of which is constituted of a probe immobilizing part, and a biochemical reaction apparatus including the same. As an example, the probe immobilizing part is formed by a detecting probe, constituted of an oligonucleotide having a known base sequence and fixed on a substrate, and the sample solution contains a biopolymer capable of performing interaction with the biopolymer of the detecting probe.

2. Description of the Related Art

There is already known an apparatus, utilizing a plurality of probe DNAs with known base sequences, for detecting presence/absence of a nucleic acid molecule executing a specific coupling with each probe DNA, namely executing a hybridization with each probe DNA. Such detection is utilized for specifying a partial sequence contained in the base sequence of the nucleic acid molecule, for detecting a target nucleic acid contained in a sample solution derived from an organism, or for identifying a genus or a species of various bacteria, based on the characteristics of gene DNA.

In order to promptly and exactly execute a hybridization with a plurality of probe DNAs, there is utilized a probe array (DNA microarray) in which a plurality of probe DNAs are regularly arrayed on a solid phase. In case of utilizing such probe array, the biopolymers in the sample solution or the like are hybridized with the probe DNAs regularly arrayed on the solid phase, thereby executing detection or quantification of the target nucleic acid in the sample solution. Such probe array allows to simultaneously detect presence/absence of a plurality of nucleic acid molecules respectively coupling with a plurality of probe DNAs. Such process is generally executed by preparing a reaction chamber at least a part of which is constituted of a substrate on which the probes are immobilized, then filling the reaction chamber with a hybridization solution which is a sample solution, and maintaining the substrate at a constant temperature for a long time.

In case of executing a hybridization utilizing a glass substrate as a sample immobilizing substrate, the hybridization solution is generally agitated for the purpose of reducing the reaction time, increasing the level of a signal after the reaction, and obtaining a uniform level thereof. For this reason, in case of executing a hybridization utilizing a probe array, there is currently employed a hybridization apparatus having an agitating function.

U.S. Pat. No. 6,238,910 describes a hybridization apparatus for a probe array. In this apparatus, the reactivity in hybridization is improved by agitating the hybridization solution (reciprocating the solution) in a reaction tank with air.

Also Japanese Patent Application Laid-open No. 2003-315337 discloses a reflux-type biochemical reaction apparatus for executing hybridization efficiently and uniformly. As shown in FIG. 17, this apparatus includes a combined member formed by superposing and tightening a first plate member 102 and a second plate member 105. The first plate member 102 is provided with a recess 103 for holding a probe substrate 101. The second plate member 105 is provided with a flow path 106 for refluxing the sample solution, a flow inlet 107, a flow outlet 108 and a projection 109 for flow alignment. The combined member formed by the first plate member 102 and the second plate member 105 is placed in a position inclined to the horizontal plane, wherein the flow inlet 107 is positioned below the flow outlet 108. The sample solution is supplied from the flow inlet 107 into the flow path 106, wherein the sample solution is refluxed.

Japanese Patent Application Laid-open No. 2003-315337 also discloses, as shown in FIG. 18, an example in which the second plate member 105 is provided with a plurality of flow inlets 107 and a plurality of flow outlets 108. In this example, the flow inlet 107 and the flow outlet 108 are provided in four units each on the plate member 105, and lines connecting the centers of the flow inlets 107 and the flow outlets 108 opposed to each other are all parallel.

Thus, in the prior technologies, there is adopted a method of agitating the hybridization solution in the reaction tank as disclosed by U.S. Pat. No. 6,238,910 or a method of refluxing the sample solution by providing the reaction chamber with the flow inlet 107, the flow outlet 108 and the flow path 106 as described by Japanese Patent Application Laid-open No. 2003-315337. Japanese Patent Application Laid-open No. 2003-315337 also discloses providing each the flow inlet 107 and the flow outlet 108 in a plurality of units, and arranging the flow inlets 107 and the flow outlets 108 in such a manner that lines connecting the centers thereof become parallel, thereby realizing a uniform flow in the flow path 106.

The probes on the probe array are regularly arranged on the plane of the substrate, but are not fully arrayed over the entire area of the substrate, and an area not containing the probes is present in an external peripheral area of the probe array. In other words, within the two-dimensional plane of the reaction chamber, the probes constituting the probe array are present rather locally.

In the relationship between each probe and a biopolymer present in the hybridization solution, the probability of causing hybridization varies significantly depending on the position in the substrate. Such situation will be explained further with reference to FIG. 19. FIG. 19 schematically illustrates a probe array 110 and an assembly 111 of the biopolymers in the hybridization solution.

In the case that the hybridization solution is not moved in the reaction chamber, the probability of hybridization is higher in a probe positioned closer to the external periphery of the probe array group 110 (for example a probe 112a shown in FIG. 19). On the other hand, the probability of hybridization is lower in a probe 112b positioned closer to the center of the probe array group 110. This is because the microscopic movement of the biopolymer in the hybridization solution is induced by the movement of the liquid molecules constituting the hybridization solution. The probe 112a present close to the external periphery of the probe array group 110 has a less number of other competing probes in capturing the biopolymer in the hybridization solution. Therefore, a number of the biopolymers capable of coupling in the hybridization solution is larger per a probe, and the hybridization is more liable to be formed. On the other hand, a probe 112b positioned close to the center of the probe array group 110 has a larger number of other competing probes in capturing the biopolymer in the hybridization solution. Therefore, a number of the biopolymers capable of coupling in the hybridization solution is smaller per a probe, and the hybridization is less liable to be formed.

### SUMMARY OF THE INVENTION

The present invention is to provide a fluid control method, a fluid control apparatus and a biochemical reaction apparatus, in which a plurality of probes provided in a reaction chamber can relatively uniformly encounter biopolymers in a sample solution without being influenced by a position in the reaction chamber.

Specifically, the present invention is to provide a fluid control method for a biochemical reaction part including a reaction chamber at least a part of which is constituted of a probe immobilizing part having a plurality of probe biopolymers immobilized thereon, and three or more ports communicating with the reaction chamber, which method including performing control for switching the inflow of a fluid into the reaction chamber and the outflow of the fluid from the reaction chamber with respect to each of the three or more ports.

According to the present invention, a fluid displacement is made possible between the reaction chamber and the three or more ports of the biochemical reaction part. Such fluid displacement can be utilized for efficiently agitating the solution to be used for a biochemical reaction, and enables a liquid such as a cleaning liquid or a gas for expelling the liquid in the reaction chamber to flow, covering uniformly the entire reaction chamber.

Further features of the present invention will become apparent from the following description of exemplary embodiments with reference to the attached drawings.

### BRIEF DESCRIPTION OF THE DRAWINGS

FIG. 1 is a system block diagram showing an operation stand-by state of a biochemical reaction apparatus in a first embodiment of the present invention.

FIG. 2 is a plan view showing a DNA chip of the biochemical reaction apparatus shown in FIG. 1.

FIG. 3 is a magnified view of a probe array of the DNA chip shown in FIG. 2.

FIG. 4A is a view showing the structure of a plate member in the biochemical reaction apparatus shown in FIG. 1.

FIG. 4B is a cross-sectional view taken in line 4B-4B of FIG. 4A.

FIG. 4C is a side view thereof.

FIG. 5 is a cross-sectional view showing a biochemical reaction part including the DNA chip shown in FIG. 2 and the plate member shown in FIGS. 4A, 4B and 4C.

FIG. 6 is a system block diagram showing, in the biochemical reaction apparatus shown in FIG. 1, a state of filling a hybridization solution into a reaction chamber.

FIG. 7 is a system block diagram showing, in the biochemical reaction apparatus shown in FIG. 1, a state of agitating the hybridization solution in the reaction chamber.

FIG. 8 is a schematic view showing, in the state shown in FIG. 7, a movement of the hybridization solution relative to the probe array.

FIG. 9 is a system block diagram showing, in the biochemical reaction apparatus shown in FIG. 1, a first cleaning state of the reaction chamber.

FIG. 10 is a system block diagram showing, in the biochemical reaction apparatus shown in FIG. 1, a second cleaning state of the reaction chamber.

FIG. 11 is a system block diagram showing, in the biochemical reaction apparatus shown in FIG. 1, a state of discharging a cleaning liquid from the reaction chamber.

FIG. 12 is a plan view showing a DNA chip of the biochemical reaction apparatus in the first embodiment of the present invention.

FIG. 13 is a magnified view of a probe array of the DNA chip shown in FIG. 12.

FIG. 14A is a plan view showing a cassette which is a biochemical reaction part including a DNA chip shown in FIG. 12 and a cassette member.

FIG. 14B is a cross-sectional view taken in the line 14B-14B of FIG. 14A.

FIG. 14C is a side view showing the cassette of FIG. 14A.

FIG. 15 is a system block diagram showing an operation stand-by state of a biochemical reaction apparatus in a second embodiment of the present invention.

FIG. 16 is a schematic perspective view showing a biochemical reaction apparatus in a third embodiment of the present invention.

FIG. 17 is a cross-sectional view showing a biochemical reaction apparatus of the prior art.

FIG. 18 is a plan view showing a plate member of a biochemical reaction apparatus of the prior art.

FIG. 19 is an explanatory view showing a relationship between probes in a probe array and a hybridization solution.

### DESCRIPTION OF THE EMBODIMENTS

In the following, embodiments of the present invention will be explained with reference to the attached drawings.

### First Embodiment

At first, a first embodiment of the present invention will be explained.

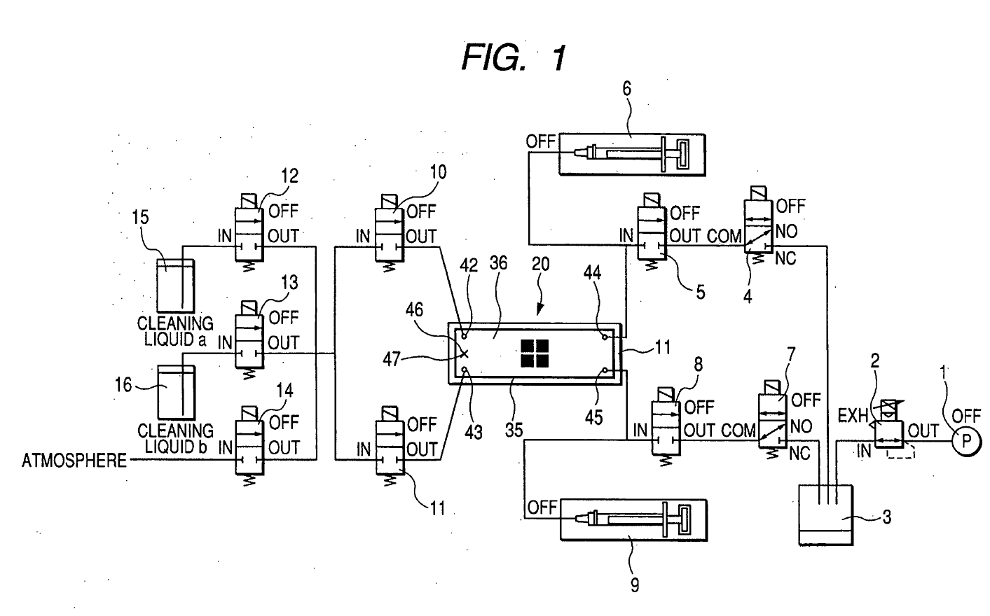

FIG. 1 is a schematic view showing an operation stand-by state of a biochemical reaction apparatus in a first embodiment of the present invention, and the biochemical reaction apparatus includes a biochemical reaction part 20 and a fluid control apparatus connected thereto.

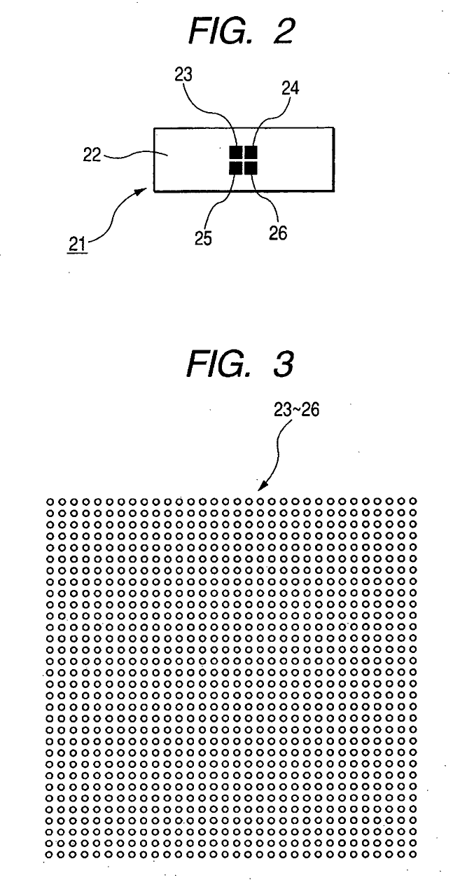

FIG. 2 is a plan view of a DNA chip 21 constituting a part of the biochemical reaction part 20. In the DNA chip 21, on a glass substrate 22 of a width of 25.4 mm, a length of 76.2 mm and a thickness of 1 mm, a plurality of probes are immobilized to constitute probe arrays 23, 24, 25 and 26. The probe arrays 23, 24, 25 and 26 are the same as one another and details of a part thereof are shown in FIG. 3. In each of the probe arrays 23, 24, 25 and 26, 1024 probes are arranged in a square shape of 32 units in the vertical direction and 32 units in the lateral direction. Each probe has a circular planar shape having a diameter of about 50 μm. The probes are arranged with a pitch of 180 μm both in the vertical and lateral directions. Each probe is formed by depositing, by an ink jet technology on the glass substrate 21, a probe biopolymer capable of hybridization with a biopolymer to be detected. As shown in FIG. 2, the four probe arrays 23, 24, 25 and 26 are arranged in a 2×2 matrix, with a spacing of 360 μm between one another.

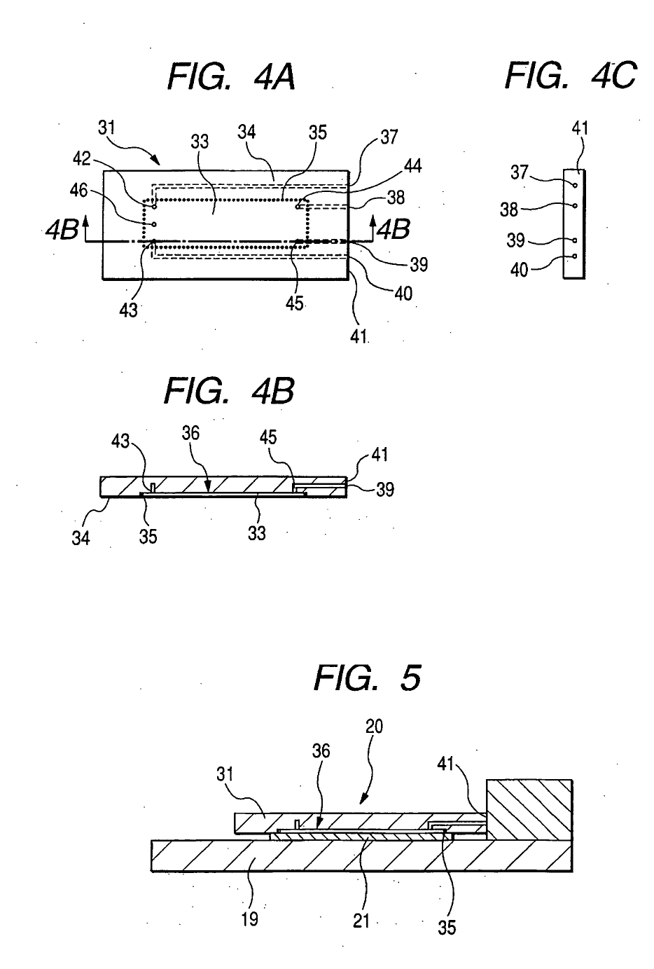

FIGS. 4A, 4B and 4C illustrate a plate member 31 which supports the DNA chip 21 and constitute the biochemical reaction part 20 together with the DNA chip 21. The plate member 31 is formed from a resin material such as polysulfone or polycarbonate. FIG. 4A is a plan view of the plate member 31; FIG. 4B is a cross-sectional view taken in the line 4B-4B of FIG. 4A; and FIG. 4C is a side view thereof.

Though not explicitly illustrated in the drawings, the plate member 31 is provided with an O-ring groove, and an internal area 33 of such O-ring groove constitutes a plane recessed by 0.1 mm from an external area 34. An O-ring 35 is fitted in the O-ring groove, and the internal area 33 of the O-ring groove 32 constitutes a reaction chamber 36, together with a probe immobilizing part (part where the probe arrays 23 to 26 are provided) of the DNA chip 21. The O-ring 35 is deformed by being pressed by the DNA chip 21, thereby sealing the reaction chamber 36 (cf. FIG. 5)

It is also possible to form the internal area 33 of the O-ring groove coplanar with the external area 34, and to add a spacer of a thickness of 0.1 mm to the external area 34, thereby forming a space constituting the reaction chamber 36.

On a lateral face 41 of the plate member 31, ports 37, 38, 39 and 40 are formed. The ports 37, 38, 39 and 40 respectively communicate, via flow paths provided in the plate member 31 (indicated by broken lines in FIG. 4A), with apertures 42, 44, 45 and 43 provided in the internal area 33 in such a manner that a fluid can flow in and flow out. The apertures 42 and 43 are positioned in the proximity of corners of the reaction chamber 36 at an upstream side, and the apertures 44 and 45 are positioned in the proximity of corners of the reaction chamber 36 at a downstream side.

Also on an upper face of the plate member 31, an aperture 46 is provided at an approximate center of the apertures 42 and 43. The aperture 46 communicates with the internal area 33 of the O-ring groove, in such a manner that a fluid can flow in and flow out. A stopper 47 (schematically illustrated in FIG. 1) is attached to the aperture 46, whereby the aperture 46 can be arbitrarily opened or closed.

The biochemical reaction apparatus of the present embodiment is principally constituted of a biochemical reaction part 20 formed of the DNA chip 21 shown in FIG. 2 and the plate member 31 shown in FIGS. 4A, 4B and 4C, and a fluid control apparatus. The fluid control apparatus includes a plurality of containers and switching control means. The switching control means executes a switching control of a fluid inflow into the reaction chamber 36, formed by the plate member 31 and the DNA chip 21, through the ports 37, 38, 39 and 40, and a fluid outflow from such reaction chamber 36.

FIG. 5 is a cross-sectional view showing the structure in the vicinity of the biochemical reaction part 20 of the present embodiment. The DNA chip 21 is set, with the probe immobilizing part at an upper side, on a temperature control table 19, and the plate member 31 is so positioned as to cover the DNA chip 21. The plate member 31 is pressurized by unillustrated pressurizing means to deform the O-ring 35, thereby fixing the DNA chip 21 and the plate member 31 in a mutually contacted state. The ports 37, 38, 39 and 40 provided on the lateral face 41 of the plate member 31 are connected, utilizing unillustrated O-rings, with the fluid control apparatus.

FIG. 1 is a system block diagram of in an operation stand-by state of the biochemical reaction part 20 and the fluid control apparatus in the biochemical reaction apparatus of the present embodiment, and the plate member 31 and the temperature control table 19 are omitted from the illustration in FIG. 1 for the purpose of clarity. The fluid control apparatus includes containers 15, 16 containing cleaning liquids a, b, and switching control means. The switching control means principally includes a vacuum pump 1, a regulator 2, a negative pressure chamber 3 formed by a hermetically sealed container, valves 4, 5, 7, 8 and 10 to 14 constituting valve means, syringe pumps 6 and 9, and flow paths formed by connecting tubes.

The components of the fluid control apparatus are connecting by tubes. More specifically, a three-way valve 4 and a two-way valve 5 are connected by a tube, and are connected, at an upstream side, to the aperture 44, and, at a downstream side, to a negative pressure chamber 3. A syringe pump 6 is connected as a branch from the pipe, connecting the aperture 44 and the two-way valve 5. Similarly, a three-way valve 7 and a two-way valve 8 are connected by a tube, and are connected, at an upstream side, to the aperture 45, and, at a downstream side, to the negative pressure chamber 3. A syringe pump 9 is connected as a branch from the pipe, connecting the aperture 45 and the two-way valve 8. The three-way valves 4 and 7 have a function of opening the flow paths, connecting the biochemical reaction part 20 and the negative pressure chamber 3, to the exterior. A two-way valve 10 is connected, at a downstream side thereof, to the aperture 42. A two-way valve 11 is connected, at a downstream side thereof, to the aperture 43. Upstream sides of the two-way valves 10, 11 are once united into a tube, which is again divided into three systems toward the upstream side, and two-way valves 12, 13 and 14 are respectively provided in these systems. The two-way valve 12 is connected, at the upstream side thereof, to a container 15 containing a cleaning liquid a, while the two-way valve 13 is connected, at the upstream side thereof, to a container 16 containing a cleaning liquid b. The two-way valve 14 is opened, at the upstream side thereof, to the exterior.

In an operation stand-by state shown in FIG. 1, the interior of the negative pressure chamber 3 is controlled by the vacuum pump 1 and the regulator 2, at a predetermined pressure (for example atmospheric pressure—30 kPa), while the valves 4, 5, 7, 8 and 10 to 14 are all closed and the pumps 1, 6 and 9 are not operated. In such state, there is no displacement of the fluid.

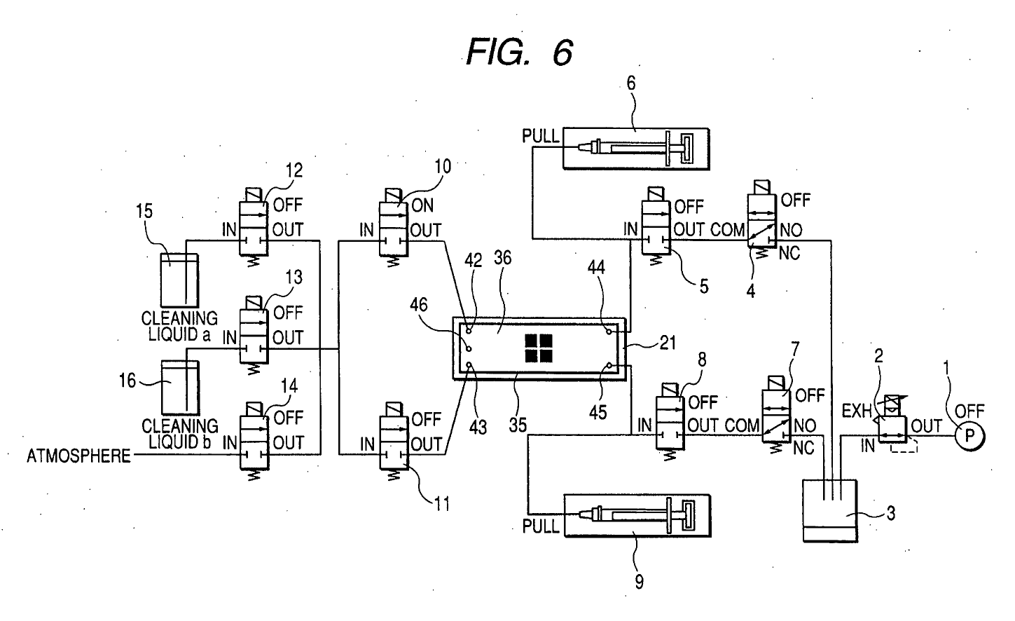

The biochemical reaction apparatus of the present embodiment executes, from the operation stand-by state shown in FIG. 1, operations shown in FIGS. 6 to 11. In the drawings, a fluid displacement is indicated by a thicker line.

FIG. 6 is a system block diagram showing an operation of filling a hybridization solution into the reaction chamber 36. The stopper 47 (cf. FIG. 1) is detached from the aperture 46, and the hybridization solution is poured by a pipette (not shown) into the aperture 46. In this operation, the syringe pumps 6, 9 execute suction operations to securely introduce the hybridization solution into the reaction chamber 36. The suction operation may be executed only in either of the syringe pumps 6 and 9, but it is preferable to execute the suction operation in both the syringe pumps 6 and 9 in order to fill the reaction chamber 36 completely with the hybridization solution, without leaving air therein. The syringe pumps 6 and 9 may be designed with such a volume as to fill the reaction chamber 36 with the hybridization solution. Otherwise, operations of the syringe pumps 6 and 9 may be controlled by a detection signal of a sensor (not shown) for monitoring the interior of the reaction chamber 36, indicating that the reaction chamber 36 is filled with the hybridization solution.

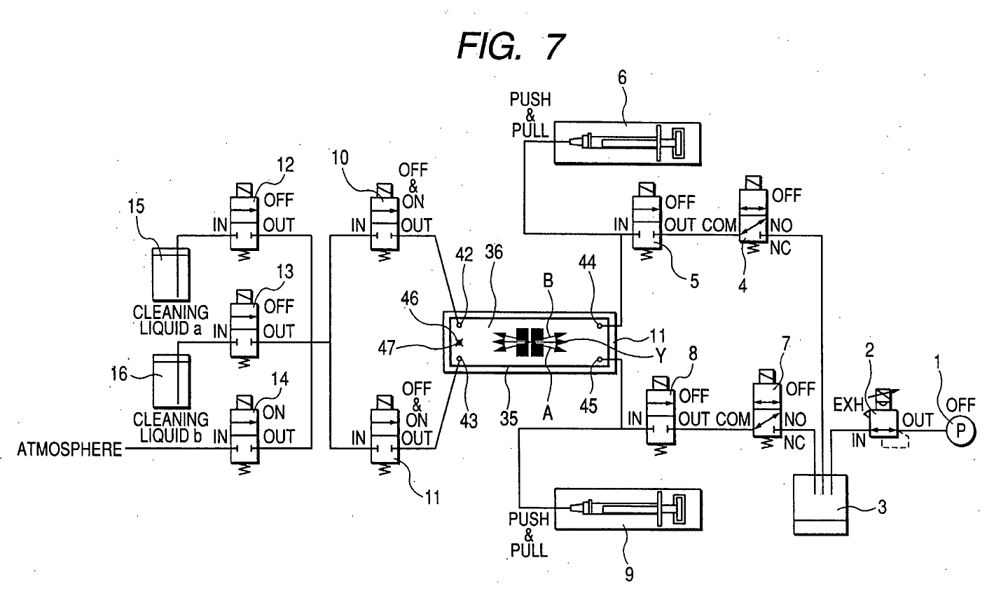

FIG. 7 is a system block diagram showing an operation of agitating the hybridization solution filled in the reaction chamber 36. The aperture 46 is closed by fitting the stopper 47. The two-way valve 14 is turned on to open the upstream side to the external air. In this state, the two-way valves 10 and 11 are turned on to connect IN and OUT, and the syringe pumps 6 and 9 are simultaneously put into push-pull operations, whereby the hybridization solution in the reaction chamber 36 executes an approximately uniform reciprocating displacement in a direction indicated by an arrow Y shown FIG. 7. Also the two-way valve 10 is turned on to connect IN and OUT, while the two-way valve 11 is turned off, and the syringe pump 9 is put into a push-pull operation while the syringe pump 6 is turned off, whereby the hybridization solution in the reaction chamber 36 executes a reciprocating displacement in a direction indicated by an arrow A shown in FIG. 7, inclined to the arrow Y. Also the two-way valve 11 is turned on to connect IN and OUT, while the two-way valve 10 is turned off, and the syringe pump 6 is put into a push-pull operation while the syringe pump 9 is turned off, whereby the hybridization solution in the reaction chamber 36 executes a reciprocating displacement in a direction indicated by an arrow B shown in FIG. 7, inclined to the arrow Y.

In the present embodiment, in the course of a hybridization reaction, the on/off operation of each of the two-way valves 10, 11 and the on/off operation of each of the syringe pumps 6, 9 are repeated in a suitable combination. Thus the reciprocating displacements of the hybridization solution in the directions indicated by the arrows Y, A and B are executed in an arbitrary combination. The hybridization is a reaction requiring about ten to several tens of minutes, or even several hours in a slower case, and, during such period, the reciprocating displacements of the hybridization solution in the aforementioned three directions (those indicated by arrows Y, A and B) are executed in a combination. By this method, the hybridization solution in the reaction chamber 36 is agitated in more different directions, in comparison with the case of reciprocating displacement in the direction Y only.

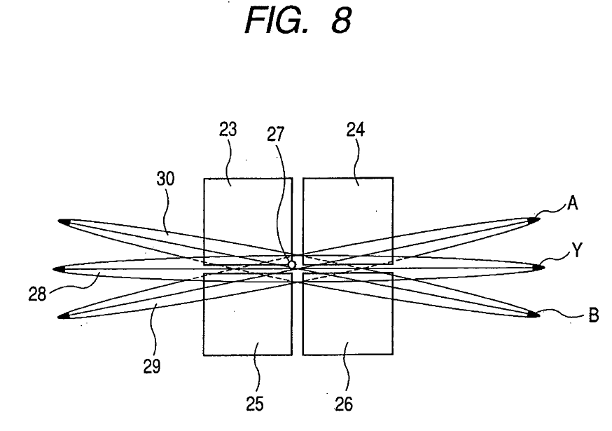

FIG. 8 is a schematic view showing the movement of the hybridization solution relative to the probe arrays 23, 24, 25 and 26. FIG. 8 illustrates, in collective manner, the directions in which the hybridization solution is moved relative to probe arrays 23, 24, 25 and 26 fixed glass substrate 22. As explained above, the hybridization solution is moved in a reciprocating motion in the directions of the arrows Y, A and B.

Now, let us consider a probe 27 positioned at the approximate center of the probe arrays 23, 24, 25 and 26. During the reciprocating displacement of the hybridization solution in the direction of the arrow Y, the probe 27 may encounter biopolymers present in an oval area 28, shown in FIG. 8. Similarly, during the reciprocating displacement of the hybridization solution in the direction of the arrow A, the probe 27 may encounter biopolymers present in an oval area 29, shown in FIG. 8. Also during the reciprocating displacement of the hybridization solution in the direction of the arrow B, the probe 27 may encounter biopolymers present in an oval area 30, represented in FIG. 8. Therefore, the probe 27 encounters more promptly a larger number of biopolymers present in a wider area of the hybridization solution, in comparison with the case of reciprocating displacement only in the direction of the arrow Y. In practice, the reciprocating displacements in the directions of arrows Y, A and B are used in combination, in suitably shifted periods. Thus the probe 27 will more promptly encounter a larger number of biopolymers present in an area of the hybridization solution, larger than the total sum of the areas 28, 29 and 30. As a result, in the case that the hybridization solution contains a biopolymer capable of hybridizing with the probe 27, the probability of succeeding in hybridization without a failure in mutual encounter becomes higher, thereby improving the precision of detection.

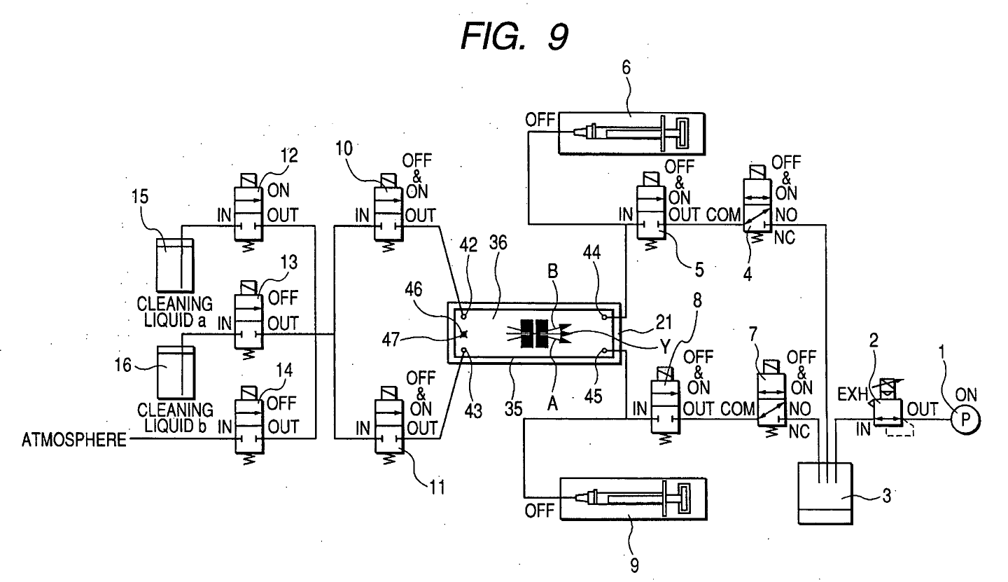

FIG. 9 is a system block diagram showing a cleaning operation after the hybridizing operation is completed. The hybridization solution filled in the reaction chamber 36 is discharged, and the cleaning liquid a in the container 15 is used to clean the interior of the reaction chamber 36, and to particularly wash off the biopolymers incompletely bonded to the probes by mismatchings, and the biopolymers deposited on the glass substrate 22. The cleaning liquid a in the present embodiment is a 2×SSC/0.1% SDS solution.

In this operation, the aperture 46 is closed by the stopper 47, as in the agitating state for the hybridization solution shown in FIG. 7. When the cleaning step is initiated, the vacuum pump 1 is turned on, whereby the interior of the negative pressure chamber 3 is controlled at a predetermined negative pressure, set by the regulator 2. Among the two-way valves 12, 13 and 14, the two-way valve 12 alone is turned on whereby the upstream side thereof communicates with the container 15 for the cleaning liquid a. In this state, the two-way valves 10 and 11 are turned on to connect IN and OUT, and the three-way valve 4, the two-way valve 5, the three-way valve 7 and the two-way valve 8 are simultaneously turned on, whereby the cleaning liquid a flows in the reaction chamber 36, approximately uniformly in a direction of an arrow Y in FIG. 9. Also when the two-way valves 10 is turned on to connect IN and OUT while the two-way valve 11 is turned off, and the three-way valve 4 and the two-way valve 5 are turned off while the three-way valve 7 and the two-way valve 8 are turned on. Then the cleaning liquid a flows in the reaction chamber 36, approximately uniformly in a direction of an arrow A, inclined from the direction of the arrow Y. Also when the two-way valve 10 is turned off and the two-way valve 11 is turned on to connect IN and OUT while the three-way valve 7 and the two-way valve 8 are turned off, and the three-way valve 4 and the two-way valve 5 are turned on, the cleaning liquid a flows in the reaction chamber 36, approximately uniformly in a direction of an arrow B, inclined from the direction of the arrow Y.

In the course of cleaning operation by the cleaning liquid a, requiring several seconds to several tens of seconds, the flows in the directions of the arrows Y, A and B are executed in an arbitrary combination, whereby the cleaning liquid a flows in more different directions in the reaction chamber 36, in comparison with the case of flow in the direction of the arrow Y only. As a result, the interior of the reaction chamber 36 can be cleaned uniformly.

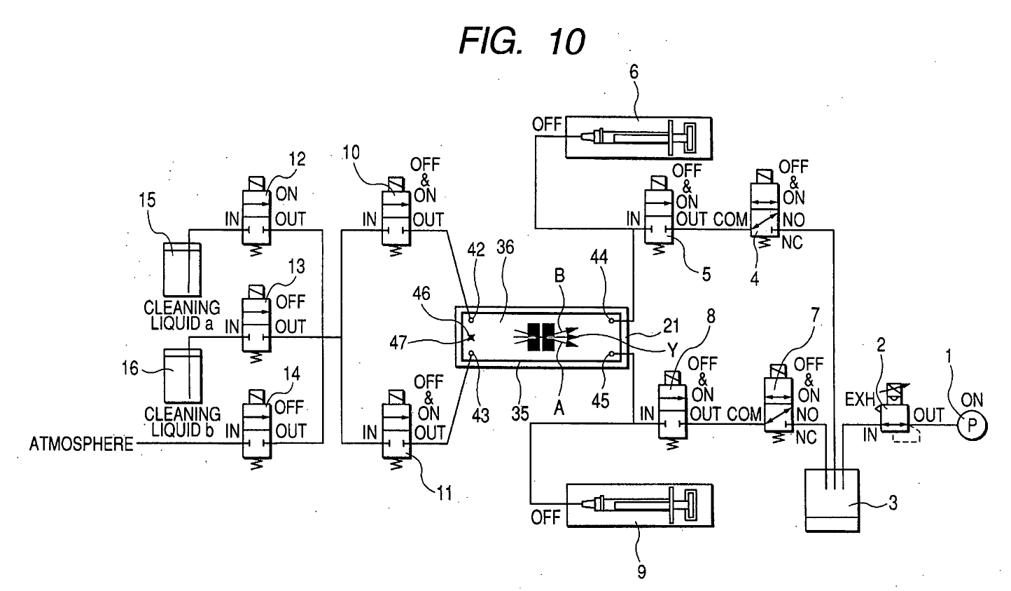

FIG. 10 is a system block diagram showing a cleaning operation with the cleaning liquid b, subsequent to the cleaning with the cleaning liquid a. The basic functions are similar to those in the above-described cleaning operation with the cleaning liquid a, except that, among the two-way valves 12, 13 and 14, the two-way valve 13 only is turned on whereby the upstream side thereof communicates with the container 16 of the cleaning liquid b. Other operations and effects are similar to those in the above-described cleaning operation with the cleaning liquid a, and will not be explained in repetition. In the present embodiment, the cleaning liquid b is purified water.

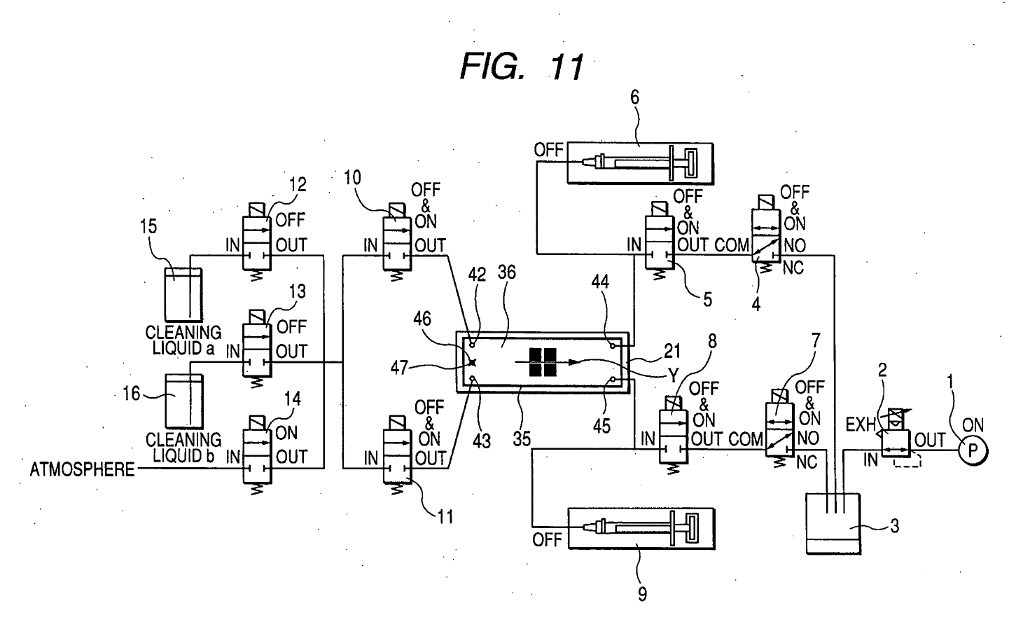

FIG. 11 is a system block diagram, showing an operation, after the cleaning with the cleaning liquid b, of discharging the cleaning liquid b filled in the reaction chamber 36. As in the agitating state of the hybridization solution shown in FIG. 7 and in the cleaning states shown in FIGS. 9 and 10, the aperture 46 is closed by fitting the stopper 47. Also the vacuum pump 1 is turned on, and the interior of the negative pressure chamber 3 is controlled at a predetermined negative pressure set by the regulator 2. Among the two-way valves 12, 13 and 14, the two-way valve 14 alone is turned on whereby the upstream side thereof communicates with the exterior. In such state the two-way valves 10 and 11 are turned on to connect IN and OUT, and the three-way valve 4, the two-way valve 5, the three-way valve 7 and the two-way valve 8 are simultaneously turned on, whereby the cleaning liquid b flows in the reaction chamber 36, approximately uniformly in a direction of the arrow Y, then passes through the aperture 44 and 45 and finally recovered in the negative pressure chamber 3. In this operation, by turning on a set of the three-way valve 4 and the two-way valve 5, and a set of the three-way valve 7 and the two-way valve 8 intentionally with a time difference therebetween, the cleaning liquid b can be securely discharged without being left in the vicinity of the apertures 44 and 45, namely in downstream corner parts of the reaction chamber 36.

In the present embodiment, as explained in the foregoing, the switching control means of the fluid control apparatus can realize a fluid displacement between the reaction chamber 36 and each of three or more ports 42 to 45 of the biochemical reaction part 20. In particular, there can be realized a continuous fluid flow from a port, through the reaction chamber, to another port. Also the fluid in the reaction chamber 36 can be made to flow not in a single direction only but in two or more directions, and such fluid displacements may be utilized for agitating the fluid in the reaction chamber 36 and for causing a fluid flow over the entire reaction chamber 36. For example, in the case that the fluid in the reaction chamber 36 is a hybridization solution, a sufficient agitation enable each probe in the probe arrays 23 to 26 of the biochemical reaction part 20 to more securely encounter the biopolymers present in the hybridization solution. As a result, regardless of the position of each probe in the probe arrays 23 to 26, the biopolymers in the hybridization solution can be supplied uniformly, so that the hybridization can be achieved more efficiently than in the prior technology. Thus, the precision is improved in processing the biopolymers in the hybridization solution (for example detection of a biochemical reaction).

Also in the case that the fluid in the reaction chamber 36 is a cleaning liquid, the aforementioned fluid displacement is utilized for causing the cleaning liquid to flow over the entire reaction chamber 36, whereby the cleaning liquid can flow more uniformly on the probe arrays 23-26 and the substrate 22 of the DNA chip 21. As a result, the cleaning operation can be executed more efficiently and more uniformly than in the prior technologies.

As explained above, the present embodiment allows to execute the hybridization and the cleaning operation in efficient and uniform manner, thereby achieving a reduction in the process time, an improvement in the level and uniformity of the signal after the reaction, and an improvement in S/N ratio between the signal from the probe and noises around the probe.

Also in case of displacing a gas such as air, as the fluid, in a state where the reaction chamber 36 is filled with a liquid such as a cleaning liquid, the gas can be made to cover the entire reaction chamber 36, thereby discharging the liquid from the reaction chamber 36 without being left therein. Thus, in detecting the probe signal, the detection is not hindered by the liquid remaining in the reaction chamber 36.

### Second Embodiment

In the following, a second embodiment of the present invention will be explained with reference to the attached drawings.

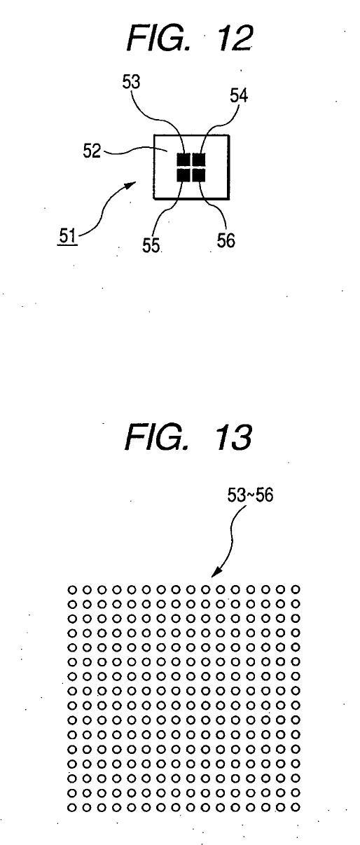

FIG. 12 is a plan view of a DNA chip 51 in the second embodiment of the present invention. In the DNA chip 51, on a glass substrate 52 of a size in vertical direction of 20 mm, a size in lateral direction of 20 mm and a thickness of 1 mm, a plurality of probes are immobilized to constitute probe arrays 53, 54, 55 and 56. The probe arrays 53, 54, 55 and 56 are the same and details of a part thereof are shown in FIG. 13. In each of the probe arrays 53, 54, 55 and 56, 256 probes are arranged in a square shape of 16 units in the vertical direction and 16 units in the lateral direction. Each probe has a circular planar shape having a diameter of about 50 μm. The probes are arranged with a pitch of 180 μm both in the vertical and lateral directions. Each probe is formed by depositing, by an ink jet technology on the glass substrate 51, a probe biopolymer capable of hybridization with a biopolymer to be detected. As shown in FIG. 12, the four probe arrays 53, 54, 55 and 56 are arranged in a 2×2 matrix, with a spacing of 360 μm between one another.

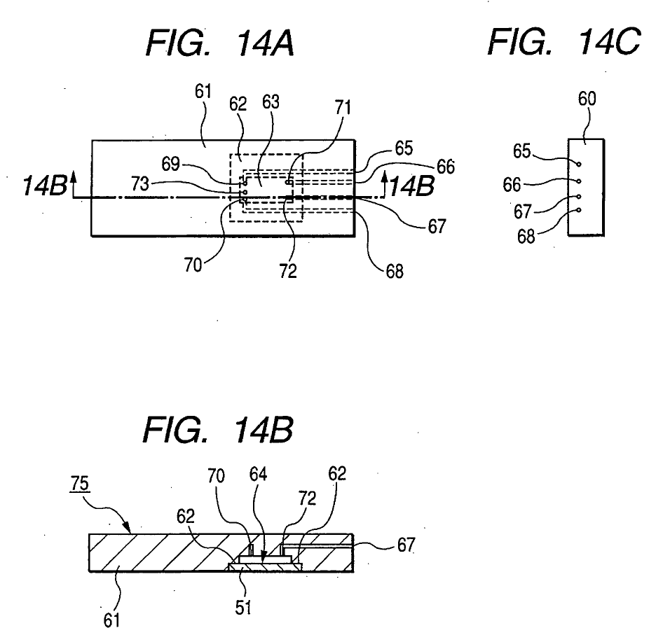

FIGS. 14A, 14B and 14C illustrate the structures of a cassette 75, constituting a biochemical reaction part and formed by integrally adhering the DNA chip 51, shown in FIG. 12, with a cassette member 61. FIG. 14A is a plan view of the cassette 75; FIG. 14B is a cross-sectional view taken in the line 14B-14B of FIG. 14A; and FIG. 14C is a lateral view thereof.

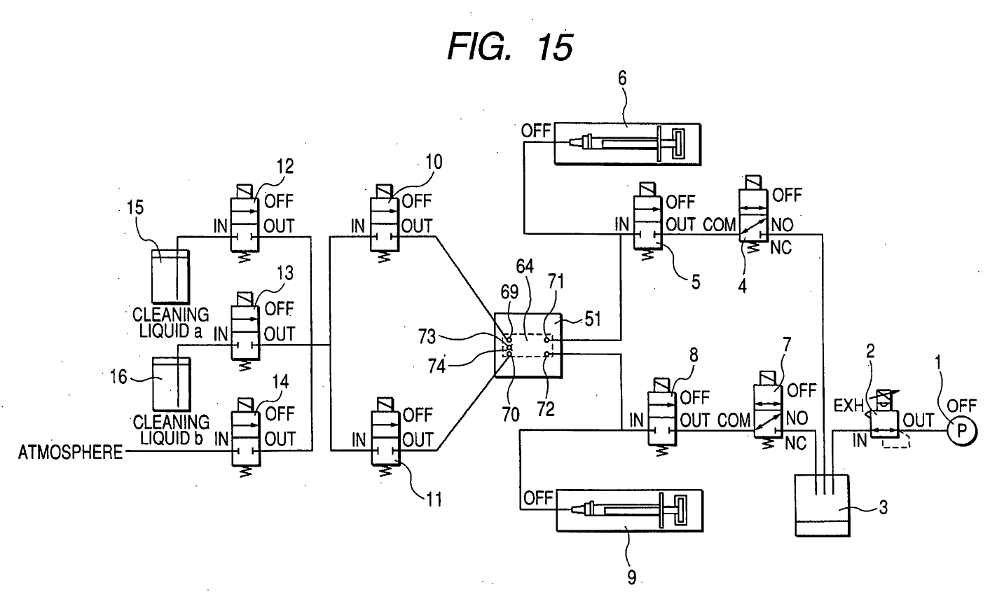

The cassette member 61 is formed by a resin material such as polysulfone or polycarbonate. The cassette member 61 is provided with an adhesion area 62 for adhering the DNA chip 51, and an internal area 63 thereof constitutes a plane recessed by 0.5 mm from the adhesion area 62. The DNA chip 51 is adhered in the adhesion area 62, and the DNA chip 51 and the area 63 inside the adhesion area constitutes a reaction chamber 64. The reaction chamber 64 has a size in vertical direction of 8 mm, a size in lateral direction of 14 mm and a height of 0.5 mm. On a lateral face 60 of the cassette member 61, ports 65, 66, 67 and 68 are formed. The ports 65, 66, 67 and 68 respectively communicate, via flow paths provided in the cassette member 61 (FIG. 14C), with apertures 69, 71, 72 and 70 provided in the reaction chamber 64 in such a manner that a fluid can flow in and flow out. The apertures 69 and 70 are positioned in the proximity of corners of the reaction chamber 64 at an upstream side, and the apertures 71 and 72 are positioned in the proximity of corners of the reaction chamber 64 at a downstream side. Also on an upper face of the cassette member 61, an aperture 73 is provided at an approximate center of the apertures 69 and 70. The aperture 73 communicates in such a manner that a fluid can flow into and out from the reaction chamber 64. A stopper 74 (schematically illustrated in FIG. 15) is attached to the aperture 73 whereby the aperture 73 can be arbitrarily opened or closed.

FIG. 15 is a system block diagram of a biochemical reaction apparatus of the present embodiment, and the cassette member 61 is not illustrated in FIG. 15 for the purpose of clarity. The biochemical reaction apparatus of the present embodiment is principally constituted of a cassette 75 constituting a biochemical reaction part formed by integrally adhering the DNA chip 51 (cf. FIG. 12) and the cassette member 16 shown in FIGS. 14A, 14B and 14C, and a fluid control apparatus. The fluid control apparatus includes containers 15, 16 and switching control means. The switching control means executes a switching control of a fluid inflow into the reaction chamber 64 of the cassette 75 through the ports 65, 66, 67 and 68, and a fluid outflow from the reaction chamber 64.

The present embodiment, as being equipped, as the biochemical reaction part, with a cassette 75 which can be mounted on or detached from the fluid control apparatus and which can be detached from the fluid control apparatus for easy handling, facilitates operations such as detection of biopolymers. It is particularly effective in case of inspecting a number of samples in succession. Other structures and operations of the fluid control apparatus, being basically same as those in the first embodiment explained above, will be represented by same symbols and will not be explained further.

### Third Embodiment

There have been explained examples of fluid control by the fluid control apparatus, on the biochemical reaction part 20 including the reaction chamber 36 in the above-explained first embodiment and on the cassette 75 including the reaction chamber 64 in the second embodiment. However, such fluid control apparatus need not be constructed separately from the reaction chamber 36 or 64. A biochemical reaction unit, integrally incorporating a fluid control apparatus in a biochemical reaction part including a reaction chamber of which at least a part is constituted of a probe immobilizing part, is also included in the biochemical reaction apparatus of the present invention. The third embodiment shows an example of such biochemical reaction unit.

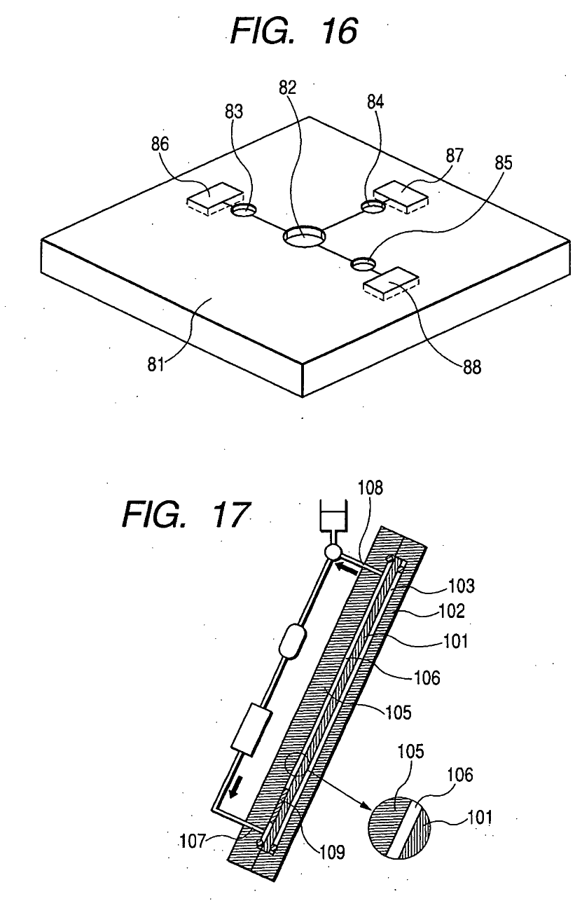

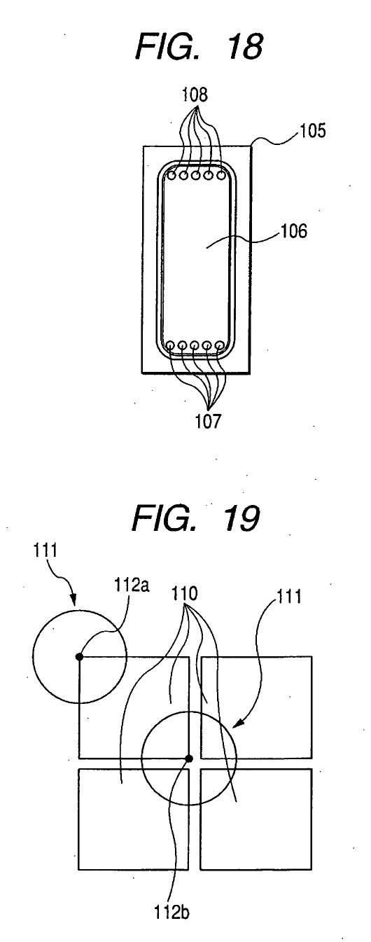

FIG. 16 is a schematic perspective view showing the structure of a biochemical reaction apparatus having a unit configuration of the present embodiment. A substrate 81 includes a reaction chamber 82, of which a part is formed by a probe immobilizing part. Around the reaction chamber 82, there are provided wells 83, 84 and 85 which communicate with the reaction chamber 82 in such a manner that fluids can flow thereinto and therefrom. The wells 83, 84 and 85 are respectively provided with fluid control apparatuses 86, 87 and 88 constituted for example of micropumps. Though not explained in detail, each of the fluid control apparatus 86, 87 and 88 includes switching control means for executing a switching control of a fluid inflow into the reaction chamber 82 and a fluid outflow from the reaction chamber 82.

In the present embodiment, a hybridization solution is poured into either one of the wells 83, 84 and 85. Then, based on a principle same as that in the first embodiment, each of the fluid control apparatus 86, 87 and 88 executes a filling into the reaction chamber 82, and generates alternately a flow directed from the reaction chamber 82 to each well and a flow from such well to the reaction chamber 82. An agitation is executed by such reciprocating displacement of the hybridization solution. It is preferable to execute, in combination, the reciprocating displacements in the directions connecting the reaction chamber 82 and the three wells 83, 84 and 85.

Similarly a cleaning liquid is poured into a well, and is filled into the reaction chamber 82 by the liquid control apparatuses 86, 87 and 88, and an overall cleaning is made possible by the reciprocating displacement of the cleaning liquid.

It is also possible to introduce air from the wells, and to expel the liquid such as the cleaning liquid in the reaction chamber 82 without remaining therein, by the fluid control apparatuses 86, 87 and 88.

As explained above, also the present embodiment can efficiently execute a filling and an agitation of the hybridization solution in the reaction chamber, a cleaning in the reaction chamber 82, and a liquid discharge from the reaction chamber 82.

### Other Embodiments

Also the liquid control apparatus in the embodiments above may be incorporated in a biochemical reaction apparatus which is capable of a series of processes from an extraction step of extracting DNA from a specimen to a detection step of detecting a hybridization reaction.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2005-348012, filed Dec. 1, 2005, which is hereby incorporated by reference herein in its entirety.

What is claimed is:

1. A fluid control method for a biochemical reaction part comprising a reaction chamber at least a part of which is constituted of a probe immobilizing part having a plurality of probe biopolymers immobilized thereon, and three or more ports communicating with the reaction chamber, which method comprising performing control of switching an inflow of a fluid into the reaction chamber and an outflow of the fluid from the reaction chamber with respect to each of the three or more ports.

2. A fluid control method according to claim 1, wherein a continuous flow of the fluid is formed so that the inflow of the fluid into the reaction chamber is performed through at least one of the three or more ports and the outflow of the fluid from the reaction chamber is performed through at least remaining one of the three or more ports.

3. A fluid control method according to claim 2, wherein the fluid is agitated by the continuous flow of the fluid.

4. A fluid control method according to claim 2, wherein the flow of the fluid directed to two or more directions is formed in the reaction chamber by the switching.

5. A fluid control method according to claim 1, wherein the control of switching is performed by valve means.

6. A fluid control method according to claim 1, wherein, as the fluid, a hybridization solution containing a biopolymer capable of coupling with the probe biopolymer, a cleaning liquid for cleaning the probe immobilizing part, or a gas is used.

7. A processing method for a biopolymer, comprising a step of introducing a hybridization solution into a reaction chamber to cause a biochemical reaction, a step of introducing a cleaning liquid into the reaction chamber to clean a probe immobilizing part, and a step of introducing a gas into the reaction chamber to discharge the liquids in the reaction chamber;
wherein a fluid control method according to claim 6 is performed in each of the steps.

8. A fluid control apparatus to be attached to a biochemical reaction part including a reaction chamber at least a part of which is constituted of a probe immobilizing part having a plurality of probe biopolymers immobilized thereon, and three or more ports communicating with the reaction chamber, the apparatus comprising a switching control means for controlling switching of an inflow of a fluid into the reaction chamber and an outflow of the fluid from the reaction chamber with respect to each of the three or more ports.

9. A fluid control apparatus according to claim 8, wherein the switching control means includes valve means.

10. A biochemical reaction apparatus comprising:
a biochemical reaction part including a reaction chamber at least a part of which is constituted of a probe immobilizing part having a plurality of probe biopolymers immobilized thereon, and three or more ports communicating with the reaction chamber so as to enable an inflow of a fluid thereinto and an outflow of the fluid therefrom; and
a fluid control apparatus according to claim 8.

11. A biochemical reaction apparatus according to claim 10, wherein the biochemical reaction part is a cassette detachably mountable on the fluid control apparatus.

12. A biochemical reaction apparatus according to claim 10, wherein the fluid control apparatus is integrally incorporated into the biochemical reaction part.
